(19) United States
(12) Patent Application Publication
(10) Pub. No.: US 2025/0221867 A1
(43) Pub. Date: Jul. 10, 2025

(54) Innovative Diaper Dispensing System with Enhanced Safety and Aesthetic Features

(71) Applicant: Michael Jay Bryant, Tampa, FL (US)

(72) Inventor: Michael Jay Bryant, Tampa, FL (US)

(21) Appl. No.: 18/636,629

(22) Filed: Apr. 16, 2024

Related U.S. Application Data
(60) Provisional application No. 63/618,417, filed on Jan. 8, 2024.

(51) Int. Cl. A61F 15/00

(52) U.S. Cl. CPC A61F 15/001

(57) ABSTRACT

The present invention discloses a diaper dispenser designed to simplify and streamline the diaper changing process. The dispenser operates like a Pez dispenser, featuring innovative design and features that are useful to parents and caregivers. This invention provides novel locking components for safety. It offers convenient access to fresh diapers, addressing common challenges during changes. Additionally, it integrates engaging aesthetic components for an enjoyable experience for all those involved.

### CROSS-REFERENCE TO RELATED APPLICATIONS

This application claims the benefit of provisional patent application Ser. No. 63/618,417 filed on Jan. 8, 2024, by the present inventor.

### 1. BACKGROUND OF THE INVENTION

The present invention addresses the practical challenges of changing a child's diapers faced by parents and caregivers multiple times a day, recognizing the need for a more convenient and safer diaper changing solution. A device that dispenses diapers to a child's parent or caregiver upon demand presents one potential solution to these challenges. Examples of such diaper dispensing devices are disclosed in U.S. Pat. No. 5,678,727 issued to Rubin et al., and U.S. Pat. No. 6,817,483 B2 to Finken et al., provide diaper storage and dispensing, but they lack innovative and practical features. For example, neither invention includes safety locks to avoid a child's unsupervised access to the spring mechanisms. In addition, the referenced inventions lack any aesthetic features despite the importance of keeping a child distracted and cooperative during the diaper changing process.

In light of these prior art limitations, this invention introduces an improved diaper dispenser device designed to efficiently manage clean diapers. The invention addresses the challenges faced by parents during diaper changes, such as accidental spills, running out of diapers, and safety concerns. In general, existing diaper dispensers often lack child locks, exposing the risk of children's fingers getting caught and harmed by the spring-loaded shelf mechanism. These devices may also lack aesthetic appeal and given that changing diapers involves a potentially disruptive infant, a diaper dispenser should include a decorative, toy-like feature. Further, practical features like a low diaper indicator will also be very useful for parents.

In response to these limitations, the current invention aims to revolutionize diaper changing by introducing a diaper dispenser that prioritizes quick and convenient diaper changes, enhances safety with child locks, improves aesthetics for use around children, and incorporates a low diaper indicator to alert parents when it's time to refill. This comprehensive solution is designed to make the diaper-changing process significantly more convenient and safer for caregivers and parents alike.

Additional objects, features, and advantages of the invention will become apparent to those skilled in the art upon consideration of the following detailed description of the preferred embodiment exemplifying the best mode of carrying out the invention as presently perceived.

### 2. SUMMARY OF THE INVENTION

The invention provides a diaper-storing and dispensing device using novel spring technology. In one embodiment, the diaper dispensing device includes a hinged door, a shelf platform, a spring mechanism, an opening at the top of said diaper housing, and a clear window located vertically along said hinged door. The hinged door joins the diaper housing using one or multiple hinged devices. The hinged door also joins the diaper housing using a latching device. The shelf platform is positioned above the spring coil for vertical up-and-down movement of the shelf platform. The spring mechanism attaches to the diaper housing.

In another embodiment, the hinged door is configured to reveal the diaper housing cavity upon opening for easy loading and retrieval of diapers.

In another embodiment, the diaper dispensing device includes one or more small rails positioned vertically adjacent to said spring mechanism are adapted to prevent lateral or improper movement of the shelf while guiding its vertical motion. In another embodiment, a lockable lid is attached to said diaper housing by one or multiple hinged devices. The lockable lid joins the diaper housing using a latching device that may be unlatched to release and detach the lockable lid. In this embodiment, the diaper dispensing device may be used without a lockable lid or alternatively, attached when desired.

In another embodiment, the lockable lid is fixedly attached by one or more hinges to the top of said diaper housing for enhancing child safety. In this embodiment, a decorative top piece may optionally be attached to said lockable lid.

In another embodiment, the lockable lid is fixedly attached by one or more hinges to the top of said diaper housing for enhancing child safety. In this embodiment, a decorative top piece is fixed to the lockable lid.

In another embodiment, the diaper dispensing device includes a decorative top piece that may optionally be attached to the lockable lid. This decorative top can be of any form and serves the purpose of entertainment.

In yet another embodiment, the diaper dispensing device includes a decorative top that may be optionally attached to the lockable lid via a male button that is fixed to the decorative top piece and is inserted into and received by a female receiver, and the female receiver is fixed to the housing body of the diaper storage and dispensing device.

In yet another embodiment, the diaper dispensing device includes a baby wipe holder that may be positioned at the top of the diaper dispensing device and locks to the housing portion of the device via a male button inserted and received by its female receiver.

In yet another embodiment, the lockable lid locks to the housing portion of the device via a male button inserted into and received by its corresponding female receiver, and the female receiver is fixed to the housing portion of said diaper dispensing device.

In another embodiment, the hinged door locks to the housing portion of the device via a male button inserted into its corresponding female receiver, and the female receiver is fixed to the housing portion of said diaper dispensing device.

In another embodiment, the top opening is the means for which a diaper is dispensed from said diaper stack, wherein a human user pulls the top diaper visible from said top opening, and said lockable top is in an unlocked position.

In another embodiment, the diaper dispensing device includes a foot pedal attached with respect to said housing toward the bottom end of said housing, and wherein downward displacement of said foot pedal causes upward hinging of said lockable lid.

In another embodiment, the diaper dispensing device includes a movable vertical stick attached to the diaper housing with means for detecting movement of the shelf for indicating the quantity of remaining diapers in said diaper stack.

In another embodiment, the body of the dispenser can be made wholly or in part of plastic, wood, synthetic wood, metal, or other rigid material.

In another embodiment, the diaper dispensing device includes a locking mechanism for said shelf platform to prevent said shelf platform from moving during the reloading of diapers, enabling convenient reloading of diapers while ensuring secure retention of the shelf in place.

In another embodiment, the diaper dispensing device is made in a size and design that is suitable for use alongside a Diaper Genie and other baby care products.

In another embodiment, the body of the diaper housing includes adjustable sides that can be modified to suit different storage needs or spatial constraints, enhancing the versatility of the dispenser's design.

In another embodiment, there is a method of simplifying diaper changing using the diaper dispensing device, comprising the steps of opening the hinged door to reveal the diaper-holding area, accessing the top diaper of the stack through the opening, observing the clear window to determine the quantity of diapers remaining, and loading additional diapers when needed and securing the lockable lid for child safety.

### 3. BRIEF DESCRIPTIONS OF THE DRAWINGS

FIG. 1 is a front view of one embodiment of the diaper dispensing device.

FIG. 2 is a partial view of the diaper housing, showing the underneath of the shelf platform, the shelf platform hook, the shelf platform band, the spring coil, and a portion of the spring.

FIG. 3 is an elevated view of the top and sides of the diaper dispensing device, showing the decorative top, the lockable lid, two sets of male and female buttons, receivers, and releases, and a low-diaper indicator.

### 4. DETAILED DESCRIPTION

For the purpose of promoting an understanding of the principles of the invention, reference will now be made to the embodiments illustrated in the drawings, and specific language will be used to describe the same. It will nevertheless be understood that no limitation of the scope of the invention is thereby intended. The described features, structures, or characteristics of the invention may be combined in any suitable manner in one or more embodiments. Such alterations and further modifications in the illustrated system, and such further applications of the principles of the invention as illustrated therein would be contemplated as would normally occur to one skilled in the art to which the invention relates. Unless otherwise defined, all technical and scientific terms used herein have the same meaning as commonly understood by one of ordinary skilled in the art. The system, methods, and examples provided herein are illustrative only and are not intended to be limiting.

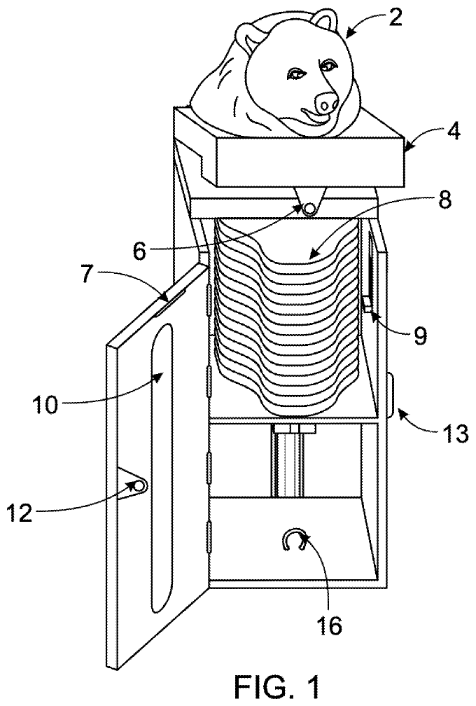

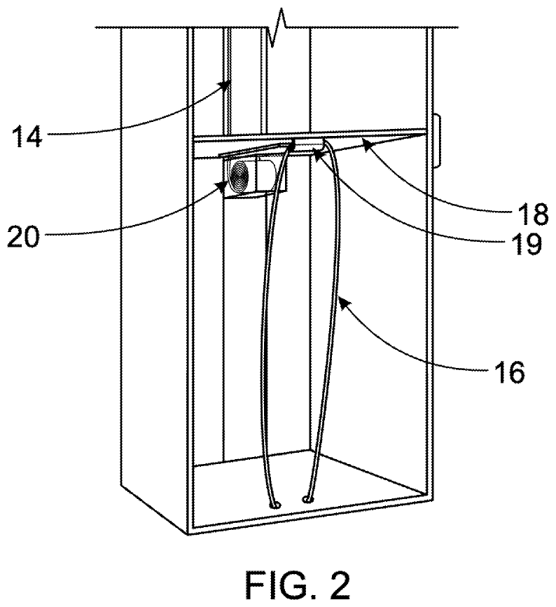

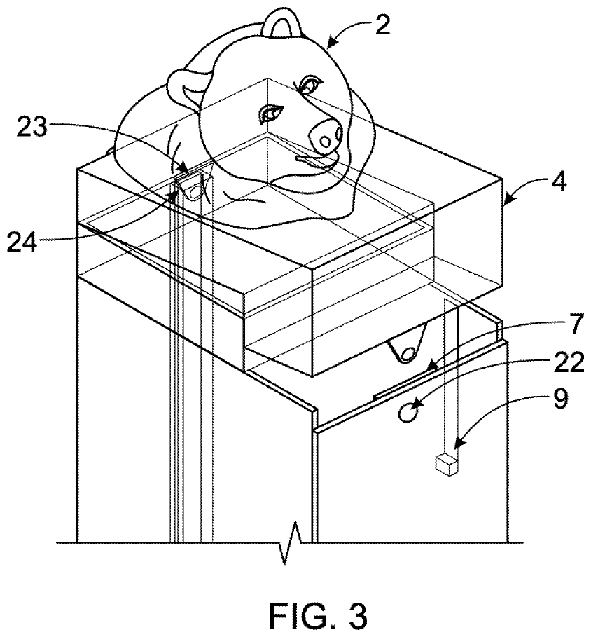

FIG. 1 is a front view of one embodiment of the diaper dispensing device. FIG. 1 illustrates a decorative top 2 that attaches to a lockable lid 4. Lockable lid 4 attaches to the diaper housing using a male button 23 (FIG. 3) that inserts into a female receiver 24 (FIG. 3). Lockable lid 4 locks to the housing using a second male button 6 that inserts into a female receiver 7. The hinged door is opened to access the diaper stack 8. The hinged door locks using a male button 12 that inserts into a female receiver 13. The hinged door includes a clear vertical window 10 for seeing the status of the diaper stack. A spring 14 attaches to the top of the diaper housing and runs vertically up and down the diaper housing. The diaper housing includes a loop for holding the shelf platform 18 (FIG. 2) in place when reloading the diaper stack 8. The diaper housing includes a diaper indicator 9.

FIG. 2 shows the lower portion of the diaper housing from a low angle. The spring attaches to the top of the housing and to the bottom of the shelf platform 18. The spring-coil 20 is positioned proximately and causes movement of the shelf platform 18. The spring-coil 20 is the spring 14 itself but turns into a spring-coil 20 in certain positions. The hook 19 hooks to the loop 16 for holding the shelf platform 18 in place. The loop 18 attaches to the bottom of the diaper housing.

FIG. 3 is an elevated view of the top and sides of the diaper dispensing device. The release button 22 unlocks the lockable lid so that it may open. The low diaper indicator 9 moves in correspondence with the number of diapers in the diaper stack to communicate how many diapers are left in the diaper stack.

1. A diaper dispensing device comprising:
a. a hinged door for accessing the diaper housing;
b. a shelf in said diaper housing for storing a diaper stack;
c. a spring mechanism(s);
d. an opening in the top of said diaper housing for easy access to top diaper of said diaper stack;
e. a clear window located vertically along said hinged door for monitoring the quantity of diapers in said diaper stack; and
f. means for joining said hinged door with diaper housing using one or multiple hinged devices and joining said lockable lid to diaper housing using one or multiple hinged devices.

2. The diaper dispensing device according to claim 1, wherein the hinged door is configured to reveal said diaper-housing cavity upon opening for easy loading and retrieval of diapers.

3. The diaper dispensing device according to claim 1, further comprising one or more small rails positioned vertically adjacent to said spring mechanism are adapted to prevent lateral or improper movement of the shelf while guiding its vertical motion.

4. The diaper dispensing device according to claim 1, further comprising an optional lockable lid that is hingedly attached to top of said diaper housing, and a decorative top may be optionally attached to said lockable lid via a male button that is fixedly attached to said decorative top and said male button may be inserted into and received by a female receiver, and said female receiver is fixed to said housing body of the diaper storage and dispensing device.

5. The diaper dispensing device according to claim 1, further comprising a lockable lid fixedly attached by one or more hinges to top of said diaper housing for enhancing child safety.

6. The diaper dispensing device according to claim 1, further comprising of a decorative top that may optionally be attached to said lockable lid.

7. The diaper dispensing device according to claim 1, further comprising of a decorative top that is fixedly attached to said lockable lid.

8. The diaper dispensing device according to claim 1, wherein the said lockable lid locks to the housing portion of the device via a male button inserted into its corresponding female receiver, and the said female receive is fixed to the housing portion of said diaper dispensing device.

9. The diaper dispensing device according to claim 1, further comprising a baby wipe holder that is optionally attached and positioned at the top of the diaper dispensing device and locks to said diaper housing via a male button inserted and received by its female receiver.

10. The diaper dispensing device according to claim 1, wherein said hinged door locks to the housing portion of the device via a male button inserted into its corresponding female receiver and the said female receiver is fixed to the housing portion of said diaper dispensing device.

11. The diaper dispensing device according to claim 1, wherein said top opening is the means for which diaper is dispensed from said diaper stack wherein human user pulls top diaper visible from said top opening and said top is in an uncovered position.

12. The diaper dispensing device according to claim 1, further comprising a foot pedal attached with respect to said housing toward a bottom end of said housing, and wherein downward displacement of said foot pedal causes upward hinging of said lockable lid.

13. The diaper dispensing device according to claim 1, wherein said spring is fixedly attached to the top of said diaper housing and fixedly attached to said shelf for moving said shelf in an upward direction.

14. The diaper dispensing device according to claim 1, wherein said spring is fixedly attached to the top of said diaper housing and is positioned proximately to said shelf and has means for causing vertical up-and-down movement to said shelf.

15. The diaper dispensing device according to claim 1, further including a movable vertical stick attached to said diaper housing with means for detecting movement of said shelf for indicating quantity of remaining diapers in said diaper stack.

16. The diaper dispensing device of claim 1, wherein the body of the dispenser can be made wholly or in part of a plastic, wood, synthetic wood, metal, or other rigid material.

17. The diaper dispensing device of claim 1, further comprising of a locking mechanism for said shelf for preventing said shelf from moving during reloading of diapers enabling convenient reloading of diapers while ensuring secure retention of the shelf in place.

18. The diaper dispensing device of claim 1, wherein being made in a size and design that is suitable for use alongside a Diaper Genie and other baby care products.

19. The diaper dispensing device according to claim 1, wherein the body of said diaper housing includes adjustable sides that can be modified to suit different storage needs or spatial constraints, enhancing the versatility of the dispenser's design.

20. A method of simplifying diaper changing using the diaper dispensing device of claim 1, comprising the steps of:
a. opening the hinged door to reveal the diaper housing;
b. accessing the top diaper of the stack through the opening;
c. observing the clear window to determine the quantity of diapers remaining; and
d. loading additional diapers when needed and securing the lockable lid for child safety.
